(19) United States
(12) Patent Application Publication
(10) Pub. No.: US 2009/0162416 A1
(43) Pub. Date: Jun. 25, 2009

(54) Polymer implants for timed release of drugs with particular emphasis on ovulation or spermiation of fish

(76) Inventors: Rex A. Dunham, Auburn, AL (US); Royall Broughton, Valley, AL (US); Amina Zuberi, Islamabad (PK); Fatma S. Kilinc-Balci, Richmond, VA (US)

Correspondence Address: HAVERSTOCK & OWENS LLP, 162 N WOLFE ROAD, SUNNYVALE, CA, 94086, US

(21) Appl. No.: 12/317,367

(22) Filed: Dec. 19, 2008

Related U.S. Application Data
(60) Provisional application No. 61/008,162, filed on Dec. 19, 2007.

(51) Int. Cl. A61L 31/16; A61P 15/08

(52) U.S. Cl. 424/426

(57) ABSTRACT

An improved cellulose acetate implant is able to more cost effectively provide ovulation of female fish. The time required for a production run of implants in CA is about 2-3 hours rather than days of the previous procedure. The method of generating the improved implant includes dissolving a hormone in water, adding acetone to the solution, adding cellulose acetate to the solution, mixing the solution, generating the CA/LHRH from the solution such as by extrusion and processing the CA/LHRH such as drying and cutting the CA/LHRH into strips of improved implants. Other polymers, substances and additives are also able to be used. The improved implants are then able to be inserted into fish at the proper time to increase ovulation which ultimately results in an increased number of fish.

### CROSS-REFERENCE TO RELATED APPLICATIONS

This application claims the benefit of U.S. Provisional Patent Application Ser. No. 61/008,162, filed Dec. 19, 2007 and entitled “CELLULOSE ACETATE IMPLANTS FOR THE OVULATION OR SPERMIATION OF FISH”; which is hereby incorporated by reference in its entirety for all purposes.

### FIELD OF THE INVENTION

The present invention relates to the field of subcutaneous polymer implants for timed-release drug delivery. More specifically, to implants for timed release of hormones in fish for increased fish reproduction.

### BACKGROUND OF THE INVENTION

Timed release of drugs is a well established technology that is used for everything from antibiotics to vitamins. Most often it is used to spread the dosage over the time it takes a pill to travel through the digestive system. Other potential controlled release sites include transdermal application and subcutaneous implants. Cellulose esters have been applied for digestive tract application. The drug chemistry and the materials (most often polymers) that control release of the drug are carefully selected according to (for example) body location, contacting fluid, osmotic pressure, pH, drug MW, drug solubility (in polymer and bodily fluids), polymer morphology, diffusion rate of drug in polymer, and geometric factors—size and shape of delivery vehicle.

One (perhaps atypical) application is the use of the drug, luteinizing hormone-releasing hormone analogue (LHRHa) to promote ovulation in seasonable ripe gravid female catfish. The induced ovulation is sometimes necessary for fish in captivity, and the knowledge of the ovulation timing is helpful to maximize the production of healthy catfish fry. The structure of LHRHa is a nine amino acid synthetic polypeptide: -Glp-His-Trp-Ser-Tyr-Gly-Leu-Arg-Pro-ethylamide-: having water, alcohol and acetone solubility and a molecular weight of about 1000. It has been found that a single injection of LHRH in the dosage of 0.45 mg/lb of body weight will induce ovulation within about 12 hours, but a two stage injection of much smaller dosage would produce similar results. The use of controlled release technology is therefore a natural consideration.

Such a procedure was developed using poly (ethylene-co-vinyl acetate), or EVAC, as the polymer, and various mixtures of bovine serum albumin (BSA) and inulin (IN) to control the release rate of LHRH. The release from EVAC, is quite slow in fish bodily fluids, and some amount of BSA/IN is typically required to provide a sufficient release rate. A syringe is used to inject an EVAC/BSA/IN/LHRH implant into the fish. The procedure for manufacturing of the implant is a mixing and molding process which is cumbersome, time consuming and incurs many potentially unneeded steps to ensure uniformity of implants.

Among the cumbersome aspects of the procedure are the facts that BSA, IN and the LHRH are insoluble in EVAC and its solvent dichloromethane. Sonication and manual grinding are suggested for obtaining a fine powder to mix in the EVAC solution, and a cold, controlled evaporation of the volatile dichloromethane is required. The procedure is a multiple day process, fraught with difficulties that a chemist would like to avoid and produce an inherently nonuniform suspension.

### SUMMARY OF THE INVENTION

An improved polymer implant, initially constructed of cellulose acetate is able to more cost effectively produce ovulation in female fish. The time required for a production run of implants in CA is about 2-3 hours rather than days of the previous procedure.

The method of generating an improved implant from (for example) a cellulose acetate (with degree of substitution of ˜1.75) includes dissolving the hormone (for example, LHRHa) in water, adding acetone to the solution, then adding polymer followed by mixing to dissolve the polymer. The solution is then extruded into a coagulation bath, a nonsolvent for the polymer (for example, toluene), to produce an elongated fiber or rod. The fiber/rod is removed from the coagulation bath and allowed to dry (solvent and coagulating nonsolvent are allowed to evaporate). A length of the rod is cut and subsequently used as a subcutaneous, time-release implant for inducing ovulation in fish.

Polymers other than cellulose acetate are able to be used for producing implants. Further, solid, implantable shapes other than a rod are able to be produced, and formation processes such as casting (followed by cutting), or molding are able to be used instead of extrusion, to produce solid polymer implants.

Some of the significant features of the invention include: the use of a polymer which is soluble in a solvent for the drug which is non-degrading; the use of a relatively hydrophilic polymer implant which allows the diffusion or elution of the drug from the implant, over time, under conditions found at the implant site; the use of typical polymer processing procedures including fiber solution extrusion technology, and in the case of wet extrusion, a suitable coagulation medium is required, for dry extrusion a rapidly evaporating solvent is required; the use of the features above to produce a time-release implant of an ovulation inducing hormone in fish and the use of the features above to produce an implant of size and shape to permit the subcutaneous injection from a syringe needle designed for that purpose.

Some other significant considerations of the invention include: extrusion of the fiber or rod is a preferred embodiment because of the semi-continuous nature of the extrusion process and the ability to control the drug dose by the cut length of fiber/rod as well as by its concentration in the implant; moderately hydrophilic polymers are preferred because most bodily fluids are aqueous; polymer implants which are insoluble in water, but soluble in mixed solvents containing water are preferred because dissolution in the body presents problems of migration and how the body disposes of such materials; polymer solvents and coagulation non-solvents should be rather innocuous common materials for both safety and economic considerations; coagulation solvents (polymer non-solvents) should ideally not be solvents for the drug being delivered, as the drug will be partially leached into the coagulation bath. This problem may be mitigated by a short residence time in the coagulation bath; selection of copolymers having variable and controllable hydrophobic groups offer a particularly elegant way of controlling the rate of drug elution into the body. Cellulose Acetate (CA) has varying degrees of substitution (DS) of the hydroxyl groups on the repeating unit. The DS of CA varies from 0 to 3 depending on the number of acetyl groups left in the polymer during manufacture. At DS=0, the polymer is pure cellulose and is insoluble in both water and acetone. At DS=3, the polymer is known as cellulose triacetate and is insoluble in water, soluble in methylene chloride and highly swollen in acetone. At DS=1.75 the polymer is insoluble in both acetone and water but soluble in certain mixtures of the two solvents. More considerations of the invention include: other polymers with compositional effects similar to CA include: carboxymethyl cellulose, other cellulose esters and ethers, copolymers of chitosan/chitin, poly(vinyl alcohol)—which like cellulose varies in the number of hydroxyl groups substituted with acetyl groups, and ionomer copolymers such as poly(ethylene-acrylic acid). Over some composition range, all of the polymers above are soluble in water at some pH between 6 and 10, and polymer composition can be modified to produce solubility and coagulation conditions in mild, relatively safe common solvents; if a coagulation non-solvent (for the polymer) is a solvent for the drug, there is concern that the drug may leach out during coagulation. The actual coagulation of the polymer tends to trap the included drug for a short time, and residence time in the coagulation bath should be minimized in these cases; diffusion enhancing additives are able to help in time-release drug dosing, and are able to be used to increase the delivery rate if needed. For water soluble drugs, hydrophilic (often water soluble) additives are used. These include, but are not limited to poly(ethylene glycol), poly(tgetrahydro furan), polymeric carbohydrates such as inulin, polypeptides such as bovine serum albumen, and water soluble compositions of polymers such as poly(vinyl alcohol). There is, of course, a preference for polymers that are biocompatible. Although the invention was developed for a particular effect (ovulation enhancement) and a particular target organism (fish), it has applications to other drugs and organisms.

Some of the advantages of the current invention over prior art include: the simplicity of manufacture of polymer implants; the uniformity of composition throughout and between implants; the ease of adjustment in drug dose; the ease of adjustment in drug elution in the body; the savings in time and materials and the recyclability of any waste material from production.

A method of generating the improved implant includes dissolving a hormone in water, adding a substance to the solution, adding a polymer such as cellulose acetate to the solution, mixing the solution, generating the CA/LHRH from the solution such as by wet extrusion, casting or molding and processing the CA/LHRH such as drying and cutting the CA/LHRH into lengths of improved implants. Other polymers, substances and additives are also able to be used. The improved implants are then able to be inserted into fish at the proper time to increase ovulation which ultimately results in an increased number of fish.

In one aspect, a fiber implant comprises a time release drug and a polymer forming the fiber implant configured for temporarily storing the time released drug, wherein both the polymer and the drug are soluble in the same solvent. The solvent is removed by casting or molding followed by evaporation, or by extrusion into a coagulating bath, removed, and dried to form a solid rod-shaped material of indeterminate length which is able to be cut to an implant length appropriate for a specific delivered dose of drug. The polymer is cellulose acetate having a degree of substitution of between 1 and 2.5 which is dissolved in a solution of acetone, water and the drug. The coagulation bath is toluene. The drug is an ovulation inducing hormone, LHRH or analogues thereof. Alternatively, the fiber implant is formed by casting a film. The film is suitable for slitting or die cutting into solid time-release drug implants.

In another aspect, a subcutaneous injected implant comprises cellulose acetate and luteinizing hormone-releasing hormone analogue.

In another aspect, a method of generating an implant comprises dissolving a hormone in water forming a solution, adding a substance to the solution of the hormone and the water, adding a polymer to the solution of the hormone, the substance and the water, mixing the solution of the polymer, the hormone, the substance and the water, generating a polymer/hormone result from the mixture of the polymer, the hormone, the substance and the water and processing the polymer/hormone result. The hormone comprises luteinizing hormone-releasing hormone analogue. The substance is a cosolvent. The substance is selected from the group consisting of acetone, alcohol, ether, Tetrahydrofuran (THF), Dimethyl formamide (DMF) and Dimethyl acetamide (DMAC). The polymer is selected from the group consisting of cellulose acetate, carboxymethyl cellulose, cellulose ethers, carbosymethyl cellulose, poly(vinyl alcohol), poly(acrylic acid copolymers), chitosan, alginate and acrylate esters. Mixing is performed using a centrifugal mixer. Mixing is performed until the polymer is dissolved. The polymer/hormone result is a solid. Generating comprises casting a film. Generating comprises pouring the mixture of the polymer, the hormone, the substance and the water into a mold and allowing the water and the substance to evaporate. Alternatively, generating comprises extruding the polymer/hormone result via a syringe pump as a fiber or rod into a coagulation bath such as a column of toluene. Alternatively, generating comprises melt extrusion. Processing comprises removing strips from the mold and cutting the strips. Alternatively, processing comprises drying and cutting the extruded polymer/hormone result. The method further comprises adding an additive to the solution wherein the additive is at least one of PEG 1000, bovine serum albumin, inulin, poly(ethylene glycol), water soluble carbohydrates, carbohydrate derivatives, and poly(THF). The substance and the polymer comprises a 20% polymer solution in a 90/10 acetone/water solvent.

In another aspect, a method of increasing ovulation in fish comprises preparing the fish and implanting a cellulose acetate/luteinizing hormone-releasing hormone analogue implant into the fish. Implanting is implemented using a syringe.

In another aspect, a system for generating an implant comprises a container, a mixing device configured for mixing a solution of the container and an extruding device configured for extruding a cellulose acetate/hormone result. The solution comprises water, acetone, cellulose acetate and a hormone. The mixing device is a centrifugal mixer. The extruding device is a syringe or other type of pump. The cellulose acetate/hormone result is extruded into a column of toluene. The hormone comprises luteinizing hormone-releasing hormone analogue. The cellulose acetate/hormone result is cut into pieces after extruding.

### BRIEF DESCRIPTION OF THE DRAWINGS

FIGS. 1-4 illustrate graphs of the release rate of tetracycline from a cellulose acetate implant.

FIG. 5 illustrates a flowchart of a method of generating an implant.

FIG. 6 illustrates a flowchart of a process of increasing ovulation in a fish.

FIG. 7 illustrates a system for producing implants.

FIG. 8 illustrates a set of implants stored in a casing.

### DETAILED DESCRIPTION OF THE PREFERRED EMBODIMENT

A simplified process of developing an implant for the ovulation or spermiation of fish is described herein. The implant is able to help induce ovulation which is sometimes necessary for fish in captivity.

A cellulose ester such as cellulose acetate (CA) is used as the control-release polymer. Cellulose acetate has a DS of 1.75, which is soluble in mixtures of acetone and water. The luteinizing hormone-releasing hormone analogue (LHRHa) is dissolved in water, then acetone is added and then the CA is added. The components are mixed in a centrifugal mixer until a homogeneous solution is obtained. The solution is poured into a mold (such as an aluminum mold) and the solvent is allowed to evaporate. The thin strips of CA/LHRH are removed from the mold and cut into lengths with the appropriate dosage. In another embodiment, wet spinning is another production method. The CA/LHRH is extruded via a syringe pump with a large bore needle into a column of toluene. A relative uniform linear density product results, although not necessarily cylindrical because of the shrinkage as the solvent leaves the fiber.

The time required for a production run of implants in cellulose acetate is about 2-3 hours rather than days by the previous procedure. The uniformity of the drug throughout the polymer is guaranteed as it is in solution. The process is easily scalable. The results for CA/LHRH as a fish implant are good. Approximately 75% of the fish produced good quality eggs. Drug release results indicate that roughly 70% of loaded tetracycline is recovered/released from a CA implant. A typical release curve appears below from a CA/tetracycline implant containing 20% of a release enhancing additive. Without the additive, a similar release extends over several weeks. There are numerous diluents that are able to be used to increase the rate of release. The ability to tailor the release profile coupled with the ease of processing the material suggests that these systems are able to be useful in applications beyond fish implants.

### Comparison of EVAC and CA

The following is a comparison of EVAC and CA for use as implants including a description of how the implants were prepared and implemented, and how the effectiveness results were obtained.

CA and EVAC-based LHRHa implants were compared for the production of channel catfish, Ictalurus punctatus, female X blue catfish, I. furcatus, hybrid embryos. EVAC based implants were more difficult to manufacture than CA implants. Fry produced per kg of female body weight were not different (P>0.05) for EVAC, 3,058 and CA, 2,621, respectively. The percent of females ovulating, latency period, fecundity and hatch were also not different for the two types of implants. Since CA implants were as efficacious as EVAC implants, and are easier to manufacture, application of CA implants would be advantageous for producing channel-blue hybrid catfish embryos.

CA is a material that is much easier to handle and is more cost effective when manufacturing LHRHa implants than EVAC by reducing labor and wastage. Additionally, CA is biodegradable, whereas EVAC is not. An inulin/BSA mixture is used as a bulking agent for both types of implants, generating channels within the solidified polymer. Upon application and contact with body fluids, the inulin/BSA mixture slowly dissolves along with the entrapped LHRHa. The rate of release of hormone is able to be changed by altering the concentration of bulking agent, inulin/BSA. Alternatively, in CA implants, it is possible to change the rate of release by altering the percent of hydroxyl group in the CA.

### Materials and Methods

### Comparison of Preparation of Implants

The EVAC procedure involves about two weeks of preparation using an ethylene/vinyl acetate copolymer as the release agent. The various steps include multiple washings of polymer pellets, dissolution in methylene chloride, dissolution of hormone/BSA and inulin in water, freeze drying, manual grinding, dispersion of the hormone mixture in the EVAC solution, vortexing, sonication, casting the solution in a mold and drying in a freezer.

The CA procedure involves dissolution of the CA in acetone/water mixed solvent, dissolving the hormone in water, mixing the two solutions (in some embodiments, the two solutions are not separate), casting a film, followed by evaporation of the solvent at room temperature and cutting the film into strips. Obviously, this procedure is simpler and quicker. Further processing simplifications are able to make the process better and produce more uniform implants. Recycling of any production waste is also easy, by simply re-dissolving the polymer in an acetone/water mixture and recasting the film.

### Spawning of Fish

All experimental fish were kept in earthen ponds throughout the year at the E. W. Fisheries Center at Auburn University. The males and females were seined and transported to tanks to be prepared for spawning. Males and females were selected by the visible reproductive readiness such as indicated by head size for the males and abdominal distention for the females.

The females for spawning were held in tanks measuring 3.0×0.47×0.61 m and with a water volume from 670-837 liters. Each tank had a constant flow through and compressed air for aeration.

The male blue catfish were brought into the laboratory and sacrificed to obtain sperm. The sperm was prepared approximately 24 hours before the females began to ovulate and then placed in the refrigerator.

### Implants

To induce ovulation, the females received a single CA or EVAC LHRHa implant. The implants were given in a single dose just behind the dorsal fin and ventrally down the body approximately 3 cm.

### Sperm Preparation

The males were weighed, sacrificed and then their testes were removed. The testes were cleaned with saline solution and trimmed with scissors to remove excess tissue and blood. The testes were weighed and placed in a clean plastic freezer bag. Then up to 10 ml of saline was added. The testes were placed in a clean plastic freezer and were mashed in the bag together with half of the total saline. The contents of the bag were then rinsed with the leftover amount of saline to bring the final solution to 10 ml of salt solution per gram of testes.

### Artificial Spawning

Ovulation occurred approximately 45 to 60 hours after implants. All females were checked for eggs every 3 hours after the first fish gave eggs. The females were checked for eggs by gently pushing on the abdomen and rubbing gently from head to tail. Bags were checked for eggs visually or by gently lifting and moving the bag to minimize the disturbance of the female. If eggs were present on the bag, the fish and the bag were removed from the water and placed in the anesthesia. When the females were giving eggs freely, they were placed in a solution with 200 ppm MS 222 and 200 ppm sodium bicarbonate until their movement slowed. The fish were then dipped into a tank of freshwater while the vent was covered by a finger to keep the eggs from leaking out while rinsing off the anesthesia. The fish was placed on a dry towel and the head was covered with a towel to catch any water leaking from the gill cavity. The fish were then taken to the stripping table and hand stripped. The eggs were stripped into pie pans greased with a thin layer of vegetable shortening to prevent sticking. The females were stripped of eggs until the eggs no longer flowed.

### Fertilization

The eggs were fertilized within minutes of the eggs being stripped and weighed. The eggs were rinsed with saline solution to remove all the blood and excess tissue from the female. If no blood was present, the eggs were not rinsed. The sperm solution was drawn into a 1 ml or 3 ml syringe. A total of 6.5×10⁷ sperm per 100 g of eggs were used for fertilization. The amount of sperm per milliliter of solution was determined by diluting the sperm sample 10 to 50 times and then determining absorbance of the sample with a spectrophotometer at 546 nanometers. A graph was constructed using regression analysis by counting the number of sperm in the undiluted solution and then running the sample through the spectrophotometer to find the frequency determining the sperm number in the serial dilutions with the spectrophotometer. The numbers were then plotted so that the number of sperm could be calculated from the spectrophotometer reading.

The eggs and sperm were gently swirled together. The eggs were transferred to an egg basket in a paddle wheel hatching trough.

### Incubation

The eggs were held in tanks with paddlewheels until hatch. The eggs treatment began 12 hours after they were fertilized. The initial treatment was always formalin (100 ppm) and then the eggs were treated three times a day, the first with copper sulfate (32 ppm), the second with formalin, and the last with copper sulfate to help prevent fungus growth until they began to hatch. The eggs were not treated between 42 to 46 hours at 28° C. after fertilization because that was found to be a critical period of development adversely affected by formalin treatments.

### Data Analysis

Percentage ovulation was calculated as number of fish giving eggs divided by total number fish for each treatment. Latency time was calculated using the number of hours from the first injection or implant to time of ovulation. The average latency was the average latency by treatment of only the fish that ovulated. Hatch was calculated by determining the number of viable embryos 12 hours prior to hatch divided by the total eggs in the original egg mass. The number of eggs per kg female body weight (relative fecundity) was determined by number of eggs spawned divided by female body weight for females that ovulated. Fry per kilogram equaled the total number of fry produced divided by the weight of the total number of females in the treatment. Egg quality was determined on a scale from 1 to 5. The score of 5 was assigned for good quality free flowing eggs with yellow color and without blood, a score of 4 indicated free flowing eggs that were sticky and with a pale yellow color, a score of 3 was assigned for free flowing eggs with clumps and blood present, a score of 2 was given to free flowing eggs containing clumps, blood and extra fluid, and a score of 1 was assigned for white eggs with excessive blood, clumps and extra fluid. Egg quality was assigned for each egg mass in the order they were stripped from the fish. The egg masses consisted of approximately 150 g of eggs.

Statistical analysis of data was conducted using Statistical Analysis System 9.1. A chi square test was calculated to find any difference in the percent ovulation for the different treatments.

### Results

Ovulation rate for EVAC implants was 87.5% and that for CA implants was 77.8% (Table 1). Latency period for EVAC implants was 52.6 hr and that for CA implants was 45.2 hr. Fecundity for EVAC implants was 11,274 and that for CA implants was 10,626. Hatch rate for EVAC implants was 31.0% and that for CA implants was 31.7%. Fry/kg for EVAC implants was 3,058 and that for CA implants was 2,600-4,200. None of these means were significantly different from one another (P>0.05).

Ovulation rate for CA implants with BSA was 33.3% and for CA implants without BSA was 100.0%. Latency period for CA implants with BSA was 38.3 hr and that for CA implants without BSA was 46.6 hr. Fecundity for CA implants with BSA was 9,422 and that for CA implants without BSA was 10,848. Hatch rate for CA implants with BSA was 48.9% and that for CA implants without BSA was 28.0%. Fry/kg for CA implants was 1,534 and that for CA implants without BSA was 3,035. None of these means were significantly different from one another (P>0.05).

TABLE
1 Ovulation %, latency period, fecundity,
hatch rate and fry/kg for channel catfish

| | | | Latency | | | |
|---|---|---|---|---|---|---|
| | | Ovulation | Period | Fecundity | Hatch | Fry/ |
| | N | % | (hr) | (eggs/kg) | % | kg |
| Pooled | | | | | | |
| Treatments | | | | | | |
| EVAC | 8 | 87.5 | 52.6 | 11,274 | 31.0 | 3,058 |
| CA | 9 | 77.8 | 45.2 | 10,626 | 31.7 | 2,621 |
| Partitioned | | | | | | |
| Treatments | | | | | | |
| EVAC | 8 | 87.5 | 52.6 | 11,274 | 31.0 | 3,058 |
| CA w/ | 3 | 33.3 | 38.3 | 9,422 | 48.9 | 1,534 |
| BSA | | | | | | |
| CA w/o | 6 | 100.0 | 46.6 | 10,840 | 28.0 | 3,035 |
| BSA | | | | | | |

### Discussion

EVAC and CA implants ovulated channel catfish females and produced hybrid fry with equal effectiveness. Based on previous results, if the same means would have been produced with greater replication, latency period would have been significantly longer for the EVAC implants. In the case of CA, implants made with BSA or without BSA were both effective, and the means that they produced for various reproductive traits were not significantly different. However, assuming that ovulation rates would not be different with greater replication, the latency period was significantly shorter for the BSA implants, and hatch rate and ultimately fry production would have been much higher for the implants with BSA compared to those without.

Previous technology used the ethylene/vinyl acetate copolymer (EVAC) dissolved in methylene chloride. Since methylene chloride is not a solvent for the hormone to be released, the solid hormone was dispersed as a powder into the solvent. Because of the minute quantity of hormone, it was necessary to dilute it with another insoluble powder in order to obtain a uniform dispersion. The process was laborious and time consuming. It was also subject to settling and nonuniformities in the production of implants. Since both the hormone and cellulose acetate (of the appropriate composition) are soluble in a mixture of acetone and water, it is possible to use solution techniques to produce the implants. Solutions, by nature are uniform and non-settling.

The initial results with CA implants indicated that they were as efficacious as EVAC implants, and were much easier to manufacture. Application of CA implants would be advantageous for producing channel-blue hybrid catfish embryos. The initial manufacturing process involved the casting of a thick cellulose acetate film and cutting the film into rectangular pieces of the appropriate size. It is also possible to extrude a fiber monofilament of appropriate diameter and cutting it to the required length. The manufacturing time for implants was reduced from many days to less than 2 days. The process was much simpler, requiring less equipment and offering less opportunity for error. The data indicate that application of CA implants would be useful for producing channel-blue hybrid catfish embryos. This new process offers potential advantages for simplicity, cost, uniformity, and possibly recycling of any production waste.

The preliminary data indicated that BSA, an additional component of the EVAC implant (used to enhance the hormone diffusion) might not be required. The diffusion rate from CA implants appears to be sufficiently rapid, and if not, is able to be adjusted by soluble additives to the CA solution or by changing the CA composition, or by soluble additives/plasticizers for the CA. The CA implants are an attractive alternative to the EVAC implants.

### Tetracycline Example

As with LHRHa implants, tetracycline was dissolved in water and then acetone and CA were added to make a 20% solution of CA in 90/10 acetone/water mixed solvent. The solution was spun from a motorized syringe through a 14 gauge needle into a hydrocarbon coagulation solvent, removed from the coagulation bath and allowed to dry overnight.

Implants were made both with and without PEG 1000 (added to increase diffusion rate and enhance release rate from the implant). The ratio of CA to PEG was 80/20. Implants were immersed in phosphate buffer, pH-6.8 at 37° C. and periodically, aliquot samples were removed and subjected to UV spectroscopy to determine the amount of tetracycline that had eluted from the implant.

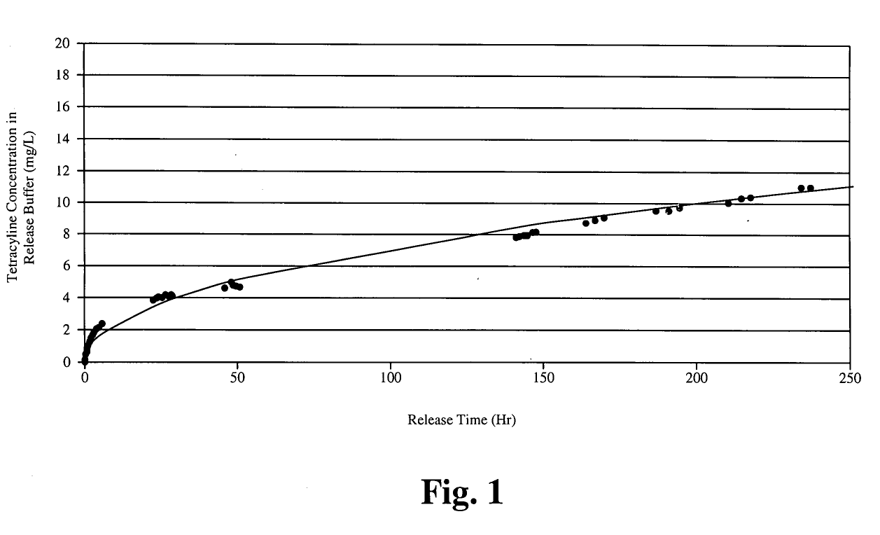

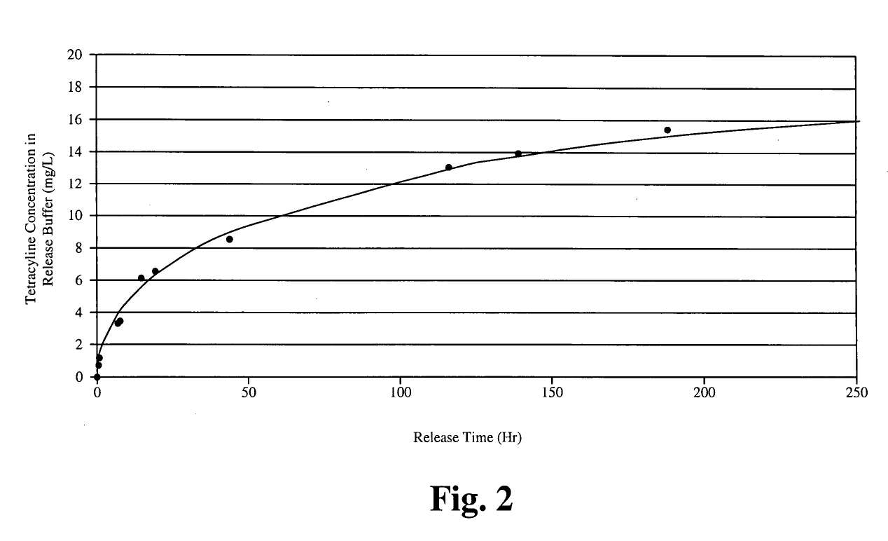

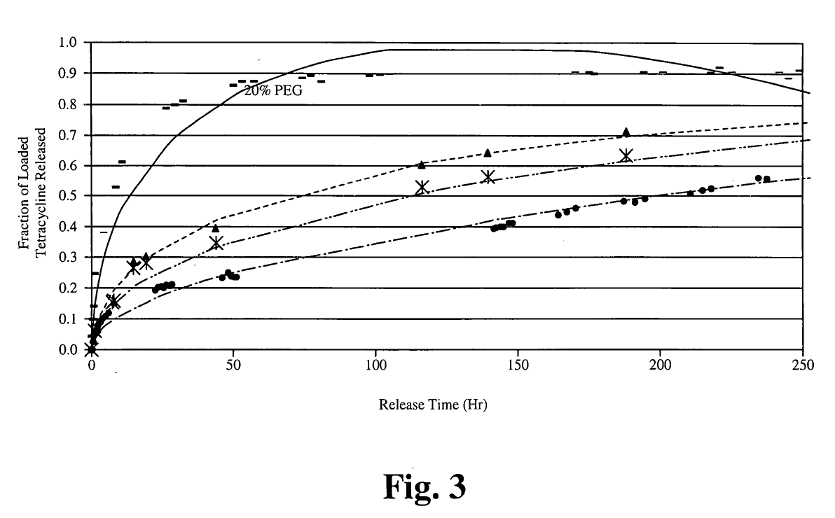

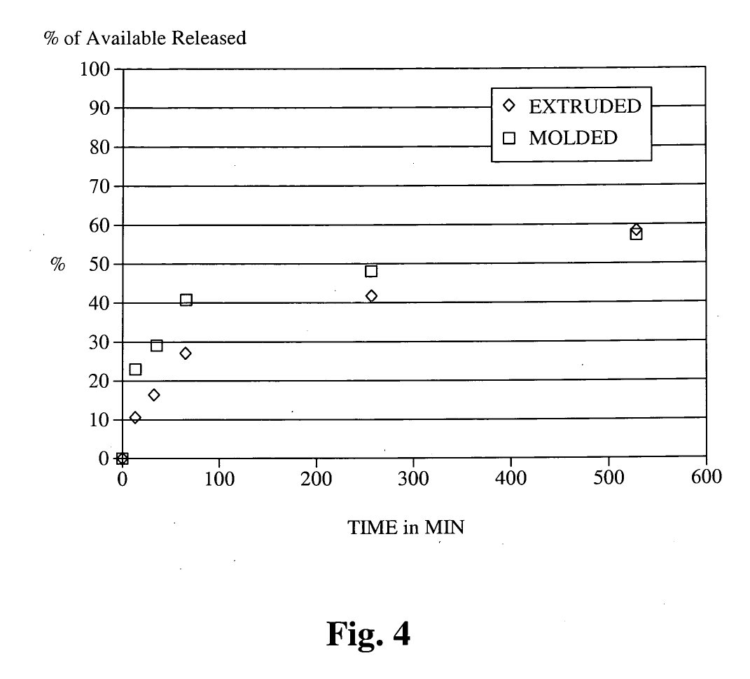

FIGS. 1-4 show graphs of the release time of Tetracycline from a CA implant. FIG. 1 is based on data without the addition of PEG. FIG. 2 is based on data where the CA is partially saponified. FIG. 3 shows the effects of the addition of PEG. FIG. 4 shows the differences of an extruded CA implant versus a molded CA implant.

Pure CA systems are able to be modeled using Fickian diffusion from a cylinder. With the PEG addition, the release rate dramatically increases. Then, the Fickian diffusion model is not applicable. Release rates are able to be controlled by the solvent composition and are likely able to be controlled by cellulose acetate degree of saponification. Nearly zero order release is able to be achieved.

### Alginate Example

A water soluble drug (0.25 g tetracycline) was dissolved 25 ml of water after which was added 2.5 g of sodium alginate. The mixture was stirred on a centrifugal mixer for about 10 minutes after which the polymer was dissolved. The solution became a cloudy viscous paste during stirring and extremely small particles of the drug had precipitated in the paste. Even so, the particles were well dispersed and exhibited no tendency to settle due to the viscosity of the paste. The paste was loaded into a syringe extruder and extruded into a coagulation bath of 1 liter of distilled water containing 20 g of calcium chloride and 2 g of 37% HCl. The paste became a gelatinous solid fiber (with sufficient strength for manufacture) in the coagulation bath and dried to a hard solid elongated fiber suitable for cutting into lengths suitable for injectable implants. The elution rate of drug from this solid was not measured.

If the solid fiber is left in the coagulation medium for an extended time (an hour or so) much of the tetracycline leaches out into the coagulation bath. As described above, a rapid coagulation is necessary if the drug is soluble in the coagulation solvent.

### Method of Generating an Implant

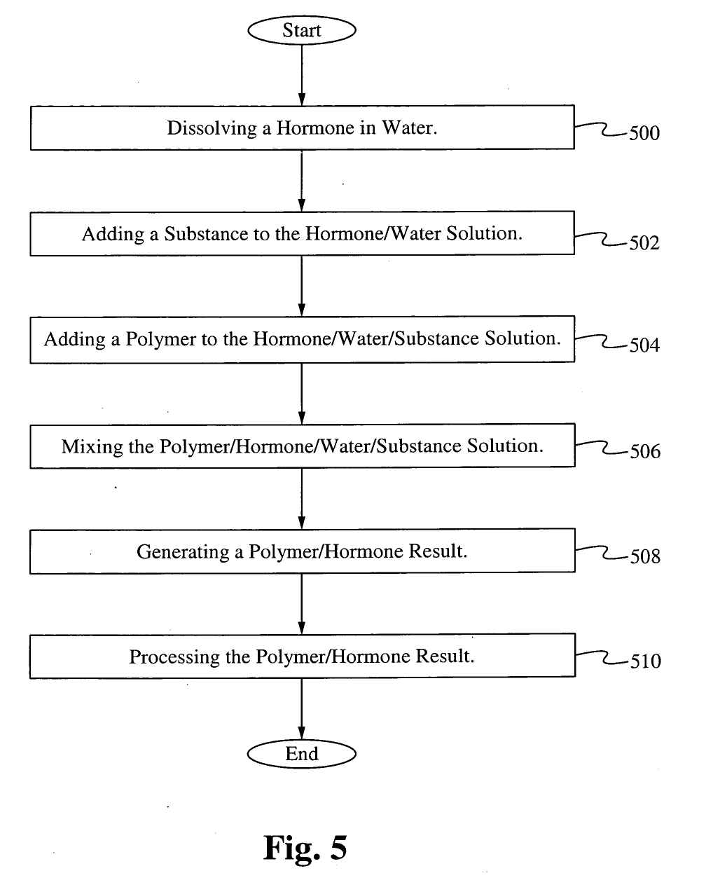

FIG. 5 illustrates a flowchart of a method of generating an implant. In the step 500, a hormone or drug is dissolved in water or a cosolvent. In some embodiments, the hormone is a Luteinizing Hormone-Releasing Hormone analogue (LHRHa). In some embodiments, PEG 1000 is also added to the water. In some embodiments, other items are added such as BSA, IN or combinations of items. In the step 502, a substance is added to the solution. In some embodiments, the substance is a cosolvent. In some embodiments, the substance is one of a ketone such as acetone, or ether, Tetrahydrofuran (THF), Dimethyl formamide (DMF), Dimethyl acetamide (DMAC) or other lower alcohols. In the step 504, a polymer such as cellulose acetate (CA) is added in with the solution. In some embodiments, a different polymer is mixed in with the solution instead of cellulose acetate. In some embodiments, adding the acetone and CA makes a 20% polymer solution in 90/10 acetone/water solvent. In the step 506, the polymer, hormone, substance and water are mixed. In some embodiments, they are mixed in a centrifugal mixer. In some embodiments, they are mixed until the polymer is dissolved. In some embodiments, the hormone is dissolved in water, the CA is dissolved in a substance/water mixed solvent separately, and then the two are mixed together. In the step 508, a polymer/hormone result is generated/regenerated. In some embodiments, the result is a solid. In some embodiments, generating includes casting a film. In some embodiments, generating includes pouring the solution into a mold (such as an aluminum mold) and the solvent is allowed to evaporate. In some embodiments, generating includes extruding the CA/LHRH into a fiber or a rod via a syringe pump or other material with a large bore needle into a coagulation bath such as a column of a material such as toluene. In some embodiments, the coagulation material is a hydrocarbon coagulation bath. In some embodiments, the coagulation material is miscible with the solvent mixture and which is able to be removed from the polymer after extrusion. In some embodiments, the drug/hormone is insoluble in the coagulating liquid to avoid leaching of the drug during coagulation. The loss of drug, if the coagulating liquid also dissolves the drug/hormone is able to be minimized by the coagulation of the polymer around it and by limiting the time in the coagulation liquid. In some embodiments, generating includes melt extrusion. In the step 510, the CA/LHRH is processed. In some embodiments, processing includes removing thin strips from of CA/LHRH from the mold and cutting the strips into lengths with the appropriate dosage. In some embodiments, processing includes drying the extruded CA/LHRH and cutting into a proper length. As is described, CA/LHRH is an example and other polymer/drug combinations are possible.

In some embodiments, the solvent used is a non-denaturing solvent, and the polymer is hydrophilic so that the drug is able to be leached from the implant through the contact with bodily fluids. Polymers which are soluble in water at mildly acidic or mildly basic conditions and which are easily precipitated by changes in pH or specific ion concentrations are also usable for controlled release matrices.

The rate of dosing in the body is controlled by the ability of bodily fluids to penetrate the implant, dissolve the drug and diffuse out of the implant and into the body (carrying the drug). In general, a more hydrophilic implant will release the drug faster. Therefore, the hydrophilicity of the polymer is able to be variable through either chemical modification of the polymer, or by the addition of a more hydrophilic additive. Among the hydrophilic additives are poly(ethylene glycol), water soluble carbohydrates and carbohydrate derivatives, inulin, BSA, poly(THF) and others. Although cellulose acetate has been described herein, others are able to be used. Carboxymethyl cellulose, cellulose ethers, carbosymethyl cellulose, poly(vinyl alcohol), chitosan, and poly(acrylic acid copolymers) are elegant candidates because of the ease of controlling the hydrophilicity by varying the concentration of hydrophobic hydrocarbon terminated side groups like acetate. Polymers such as chitosan and alginate are able to be useful because of their biocompatibility. More hydrophobic polymers such as acrylate esters are able to be useful for very slow rates of elution are desired. Many of these are soluble in rather innocuous solvents like water, ketones (acetone), alcohols and ethers, or mixtures thereof.

### Method of Implanting an Implant

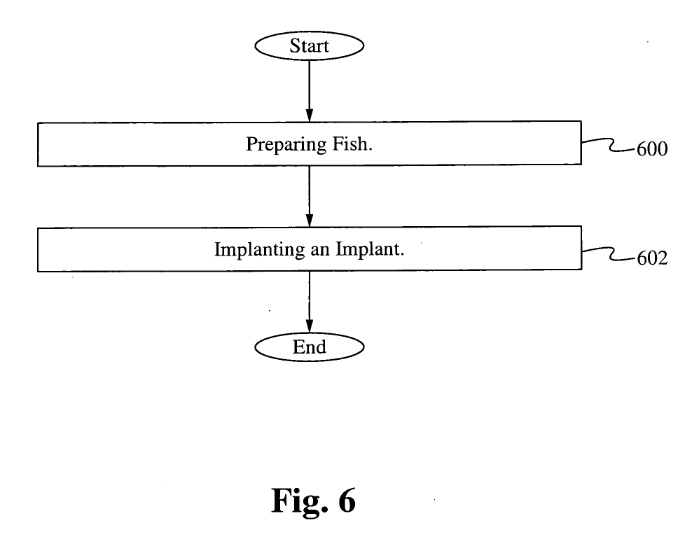

FIG. 6 illustrates a flowchart of a process of increasing ovulation in a fish. In the step 600, the fish is prepared. In some embodiments, preparing includes capturing. In the step 602, an implant is implanted in the fish. In some embodiments, the implant is implanted in a single dose behind a dorsal fin and ventrally down the body approximately 3 cm. In some embodiments, the implant is implanted using a syringe. In some embodiments, another implantation device is used.

Implant System

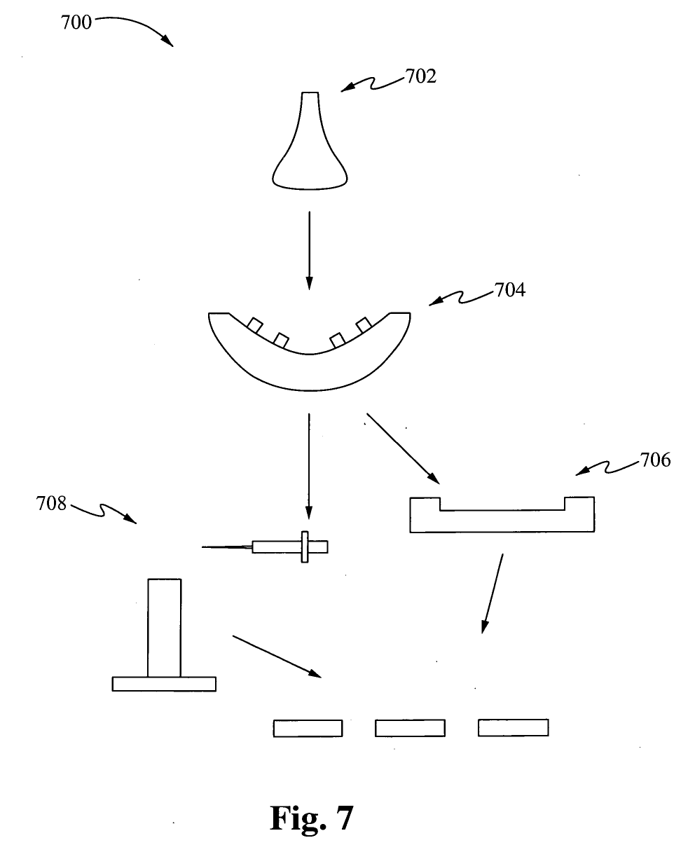

FIG. 7 illustrates a system 700 for producing implants. A container 702 such as a beaker, test tube or other object is able to be used to receive the water, substance, hormone and polymer such as cellulose acetate which together form a solution. A mixer 704 is then used to mix the solution. In some embodiments, the mixer mixes the solution in the container 702, and in some embodiments, the solution is stored in another entity when mixing. The mixer 704 is able to be a centrifugal mixer or any other type of mixing device. In some embodiments, after the solution is mixed, the solution is poured into a mold 706 where the solvent is allowed to evaporate. In some embodiments, the CA/LHRH is extruded into a fiber or a rod via a syringe pump 708 or other pump into a coagulation bath such as a column of a substance such as toluene. In some embodiments, the needle of the syringe is a 14 gage needle. Then, depending on how the CA/LHRH is obtained, either by mold or extrusion, strips or pieces are cut to the proper length with the appropriate dosage. Fewer or additional components are able to be included in the system. The above system is merely an exemplary system.

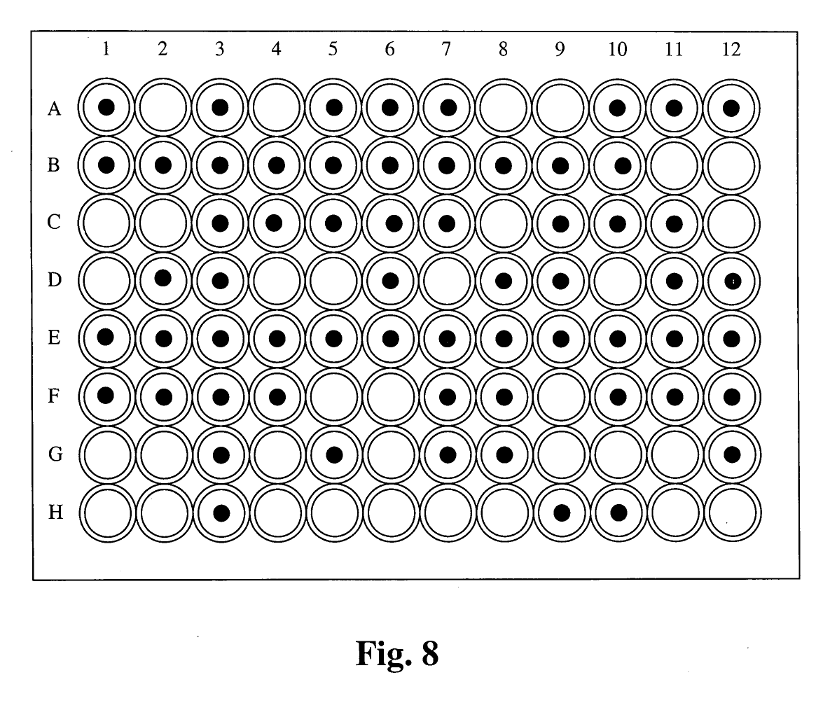

FIG. 8 illustrates a set of implants stored in a casing. In some embodiments, the implants are CA/LHRH. In some embodiments, the implants comprise a different polymer including, but not limited to, carboxymethyl cellulose, cellulose ethers, carbosymethyl cellulose, poly(vinyl alcohol), chitosan, and poly(acrylic acid copolymers), chitosan, alginate and acrylate esters. In some embodiments, the implants comprise additional materials including, but not limited to, poly(ethylene glycol), water soluble carbohydrates and carbohydrate derivatives, inulin, BSA, poly(THF) and others.

To utilize the improved implant, first of all, a method of generating the improved implant is used. The method of generating the improved implant includes dissolving a hormone in water, adding a substance such as acetone to the solution, adding cellulose acetate to the solution, mixing the solution, generating the CA/LHRH from the solution such as by extrusion and processing the CA/LHRH such as drying and cutting the CA/LHRH into strips of improved implants. As described other additives, substances and polymers are able to be used. The improved implants are then able to be inserted into fish at the proper time to increase ovulation which ultimately results in an increased number of fish.

In operation, an improved implant is able to more cost effectively provide ovulating channel catfish females or other fish. The time required for a production run of implants in CA is about 2-3 hours rather than days of the previous procedure. The uniformity of the drug throughout the polymer is guaranteed because it is in solution. The process is easily scalable to the extent needed for this low volume, high valued specialty product. The results for CA/LHRH as a fish implant are good. There are also numerous diluents that are able to be used to increase the rate of release.

Some of the significant features of the invention include: the use of a polymer which is soluble in a solvent for the drug which is non-degrading; the use of a relatively hydrophilic polymer implant which allows the diffusion or elution of the drug from the implant, over time, under conditions found at the implant site; the use of typical polymer processing procedures including fiber solution extrusion technology, and in the case of wet extrusion, a suitable coagulation medium is required, for dry extrusion a rapidly evaporating solvent is required; the use of the features above to produce a time-release implant of an ovulation inducing hormone in fish and the use of the features above to produce an implant of size and shape to permit the subcutaneous injection from a syringe needle designed for that purpose.

Some other significant considerations of the invention include: extrusion of the fiber or rod is a preferred embodiment because of the semi-continuous nature of the extrusion process and the ability to control the drug dose by the cut length of fiber/rod as well as by its concentration in the implant; moderately hydrophilic polymers are preferred because most bodily fluids are aqueous; polymer implants which are insoluble in water, but soluble in mixed solvents containing water are preferred because dissolution in the body presents problems of migration and how the body disposes of such materials; polymer solvents and coagulation non-solvents should be rather innocuous common materials for both safety and economic considerations; coagulation solvents (polymer non-solvents) should ideally not be solvents for the drug being delivered, as the drug will be partially leached into the coagulation bath. This problem may be mitigated by a short residence time in the coagulation bath; selection of copolymers having variable and controllable hydrophobic groups offer a particularly elegant way of controlling the rate of drug elution into the body. Cellulose Acetate (CA) has varying degrees of substitution (DS) of the hydroxyl groups on the repeating unit. The DS of CA varies from 0 to 3 depending on the number of acetyl groups left in the polymer during manufacture. At DS=0, the polymer is pure cellulose and is insoluble in both water and acetone. At DS=3, the polymer is known as cellulose triacetate and is insoluble in water, soluble in methylene chloride and highly swollen in acetone. At DS=1.75 the polymer is insoluble in both acetone and water but soluble in certain mixtures of the two solvents. More considerations of the invention include: other polymers with compositional effects similar to CA include: carboxymethyl cellulose, other cellulose esters and ethers, copolymers of chitosan/chitin, poly(vinyl alcohol)—which like cellulose varies in the number of hydroxyl groups substituted with acetyl groups, and ionomer copolymers such as poly(ethylene-acrylic acid). Over some composition range, all of the polymers above are soluble in water at some pH between 6 and 10, and polymer composition can be modified to produce solubility and coagulation conditions in mild, relatively safe common solvents; if a coagulation non-solvent (for the polymer) is a solvent for the drug, there is concern that the drug may leach out during coagulation. The actual coagulation of the polymer tends to trap the included drug for a short time, and residence time in the coagulation bath should be minimized in these cases; diffusion enhancing additives are able to help in time-release drug dosing, and are able to be used to increase the delivery rate if needed. For water soluble drugs, hydrophilic (often water soluble) additives are used. These include, but are not limited to poly(ethylene glycol), poly(tgetrahydro furan), polymeric carbohydrates such as inulin, polypeptides such as bovine serum albumen, and water soluble compositions of polymers such as poly(vinyl alcohol). There is, of course, a preference for polymers that are biocompatible. Although the invention was developed for a particular effect (ovulation enhancement) and a particular target organism (fish), it has applications to other drugs and organisms.

Some of the advantages of the current invention over prior art include: the simplicity of manufacture of polymer implants; the uniformity of composition throughout and between implants; the ease of adjustment in drug dose; the ease of adjustment in drug elution in the body; the savings in time and materials and the recyclability of any waste material from production.

Although the description above specifies fish, the method and improved implant described herein is able to apply to any animal or object.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A fiber implant comprising:
a. a time release drug; and
b. a polymer forming the fiber implant configured for temporarily storing the time released drug, wherein both the polymer and the drug are soluble in a same solvent.

2. The fiber implant of claim 1 wherein the solvent is removed by casting or molding followed by evaporation, or by extrusion into a coagulating bath, removed, and dried to form a solid rod-shaped material of indeterminate length which is able to be cut to an implant length appropriate for a specific delivered dose of drug.

3. The fiber implant of claim 2 wherein the polymer is cellulose acetate having a degree of substitution of between 1 and 2.5 which is dissolved in a solution of acetone, water and the drug.

4. The fiber implant of claim 2 wherein the coagulation bath is toluene.

5. The fiber implant of claim 2 wherein the drug is an ovulation inducing hormone, LHRH or analogues thereof.

6. The fiber implant of claim 1 wherein the fiber implant is formed by casting a film.

7. The fiber implant of claim 6 wherein the film is suitable for slitting or die cutting into solid time-release drug implants.

8. A subcutaneous injected implant comprising:
a. cellulose acetate; and
b. luteinizing hormone-releasing hormone analogue.

9. A method of generating an implant comprising:
a. dissolving a hormone in water forming a solution;
b. adding a substance to the solution of the hormone and the water;
c. adding a polymer to the solution of the hormone, the substance and the water;
d. mixing the solution of the polymer, the hormone, the substance and the water;
e. generating a polymer/hormone result from the mixture of the polymer, the hormone, the substance and the water; and
f. processing the polymer/hormone result.

10. The method of claim 9 wherein the hormone comprises luteinizing hormone-releasing hormone analogue.

11. The method of claim 9 wherein the substance is a cosolvent.

12. The method of claim 9 wherein the substance is selected from the group consisting of acetone, alcohol, ether, Tetrahydrofuran (THF), Dimethyl formamide (DMF) and Dimethyl acetamide (DMAC).

13. The method of claim 9 wherein the polymer is selected from the group consisting of cellulose acetate, carboxymethyl cellulose, cellulose ethers, carbosymethyl cellulose, poly(vinyl alcohol), poly(acrylic acid copolymers), chitosan, alginate and acrylate esters.

14. The method of claim 9 wherein mixing is performed using a centrifugal mixer.

15. The method of claim 9 wherein mixing is performed until the polymer is dissolved.

16. The method of claim 9 wherein the polymer/hormone result is a solid.

17. The method of claim 9 wherein generating comprises casting a film.

18. The method of claim 9 wherein generating comprises pouring the mixture of the polymer, the hormone, the substance and the water into a mold and allowing the water and the substance to evaporate.

19. The method of claim 9 wherein generating comprises extruding the polymer/hormone result via a syringe pump into a column of toluene.

20. The method of claim 9 wherein generating comprises melt extrusion.

21. The method of claim 18 wherein processing comprises removing strips from the mold and cutting the strips.

22. The method of claim 19 wherein processing comprises drying and cutting the extruded polymer/hormone result.

23. The method of claim 9 further comprising adding an additive to the solution wherein the additive is at least one of PEG 1000, bovine serum albumin, inulin, poly(ethylene glycol), water soluble carbohydrates, carbohydrate derivatives, and poly(THF).

24. The method of claim 9 wherein the substance and the polymer comprises a 20% polymer solution in a 90/10 acetone/water solvent.

25. A method of increasing ovulation in fish comprising:
a. preparing the fish; and
b. implanting a cellulose acetate/luteinizing hormone-releasing hormone analogue implant into the fish.

26. The method of claim 25 wherein implanting is implemented using a syringe.

27. A system for generating an implant comprising:
a. a container;
b. a mixing device configured for mixing a solution of the container; and
c. an extruding device configured for extruding a cellulose acetate/hormone result.

28. The system of claim 27 wherein the solution comprises water, acetone, cellulose acetate and a hormone.

29. The system of claim 27 wherein the mixing device is a centrifugal mixer.

30. The system of claim 27 wherein the extruding device is a syringe pump.

31. The system of claim 27 wherein the cellulose acetate/hormone result is extruded into a column of toluene.

32. The system of claim 27 wherein the hormone comprises luteinizing hormone-releasing hormone analogue.

33. The system of claim 27 wherein the cellulose acetate/hormone result is cut into pieces after extruding.
